(19) United States
(12) Patent Application Publication
(10) Pub. No.: US 2008/0081332 A1
(43) Pub. Date: Apr. 3, 2008

(54) Methods and devices for conducting diagnostic testing

(76) Inventor: Jun Amano, Hillsborough, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/542,476

(22) Filed: Oct. 3, 2006

(51) Int. Cl. C12Q 1/68; G01N 33/53; C12Q 1/66; G06F 19/00; C12M 3/00

(52) U.S. Cl. 435/6; 435/7.1; 435/8; 435/287.2; 702/20; 977/924

(57) ABSTRACT

The present invention is directed to methods and apparatus for analyzing a sample for the presence of one or more analytes. The sample is contacted with a channel comprising (i) a plurality of features wherein each of the features comprises a binding partner for one of the respective analytes and (ii) a plurality of silicon CMOS sensors, each of the sensors being optically coupled to a corresponding feature. The contacting is carried out under conditions for binding of an analyte to a respective binding partner. The analytes are treated to introduce a luciferase prior to or after the contacting. The luciferase has a brightness that is at least 100 times greater than firefly luciferase. Light emitted at each of the features is detected by means of the silicon CMOS sensors. The amount of light emitted at each of the features is related to the presence and/or amount of an analyte in the sample.

### BACKGROUND

The present invention relates to methods and apparatus for carrying out highly sensitive analyses for materials of interest and, more particularly, for carrying out such analyses in channels of a microfluidic system.

The clinical diagnostic field has seen a broad expansion in recent years, both as to the variety of materials of interest that may be readily and accurately determined, as well as the methods for the determination. Convenient, reliable and non-hazardous means for detecting the presence of low concentrations of materials in liquids is desired. Some materials of interest may be present in body fluids in concentrations below 10⁻¹² molar. The difficulty of detecting the presence of these materials in low concentrations is enhanced by the relatively small sample sizes that can be utilized.

The need to determine multiple analytes in biological fluids has become increasingly apparent in many branches of medicine. In endocrinology the knowledge of plasma concentration of a number of different hormones is often required to resolve a diagnostic problem or a panel of markers for a given diagnosis where the ratios could assist in determining disease progression. Other areas of interest include, for example, cancer antigen screening, allergy testing, screening of transfused blood for viral contamination or genetic diagnosis and so forth.

Any one of a number of infectious agents may cause some pathological disease states. In other cases the diagnosis and assessment of disease states may be best evaluated by the measurement of a number of analytes in a sample, such as a panel of cytokines and chemokines, a panel of tissue specific disease markers, a panel of diagnostic antibodies and antigens and the like. Another example for the utility of simultaneous analysis of multi-analytes is the determination of the level of expression of a panel of genes in a given cell population or the simultaneous detection and quantification of multiple nucleic acid sequences in a single sample. Other benefits of simultaneous detection and quantification of multiple analytes are the potential increase in throughput of the analysis and the ability to incorporate internal controls to the test sample.

Microfluidic systems have been developed for performing chemical, clinical, and environmental analysis of chemical and biological specimens. The term microfluidic system refers to a system or device having a network of chambers connected by channels, in which the channels have microscale features, that is, features too small to examine with the unaided eye, e.g., having at least one cross-sectional dimension in the range from about 0.1 μm to about 1 mm. Such microfluidic systems are often fabricated using photolithography, wet chemical etching, and other techniques similar to those employed in the semiconductor industry. The resulting devices can be used to perform a variety of sophisticated chemical and biological analytical techniques.

It is desirable to provide structures, systems, and methods that provide highly sensitive, low cost analyses for point of care applications as well as for diagnostic instrumentation.

### SUMMARY

One embodiment of the present invention is directed to a method for analyzing a sample for the presence of one or more analytes. The sample is contacted with a channel comprising (i) a plurality of features wherein each of the features comprises a binding partner for one of the respective analytes and (ii) a plurality of silicon CMOS sensors, each of the sensors being optically coupled to a corresponding feature. The contacting is carried out under conditions for binding of an analyte to a respective binding partner. The analytes are treated to introduce a luciferase prior to or after the contacting. The luciferase has a brightness that is at least 100 times greater than firefly luciferase. Light emitted at each of the features is detected by means of the silicon CMOS sensors. The amount of light emitted at each of the features is related to the presence and/or amount of an analyte in the sample.

Another embodiment of the invention is a method for analyzing a sample for the presence of one or more analytes. The sample is contacted with a channel of a microfluidic system. The channel comprises (i) a plurality of features wherein each of the features comprises a binding partner for one of the respective analytes and (ii) a plurality of silicon CMOS sensors, each of the sensors being optically coupled to a corresponding feature. The contacting is carried out under conditions for binding of an analyte to a respective binding partner. The analytes are treated to introduce a Gaussia luciferase prior to or after the contacting. Light emitted at each of the features is detected by means of the silicon CMOS sensors. The amount of light emitted at each of the features is related to the presence and/or amount of an analyte in the sample. In some embodiments the silicon CMOS sensors comprise metallic nanoparticles.

Another embodiment of the present invention is a device for analyzing a sample for the presence of one or more analytes. The device comprises (a) a channel comprising (i) a plurality of features wherein each of the features comprises a binding partner for one of the respective analytes and wherein one or more analytes that each comprises a bioluminescent marine luciferase, which has a brightness that is at least 100 times greater than firefly luciferase, are bound to a respective binding partner and (ii) a plurality of silicon CMOS sensors, each of the sensors being optically coupled to a corresponding feature, and (b) a mechanism for correlating light detected by the silicon CMOS sensors to the presence and/or amount of an analyte in the sample.

Another embodiment of the present invention is an apparatus comprising (a) a device as discussed above, (b) a computer system for controlling mechanism for correlating light detected by the silicon CMOS sensors to the presence and/or amount of an analyte in the sample, and (c) a computer program on a computer readable medium for controlling the computer system.

### BRIEF DESCRIPTION OF THE DRAWINGS

The following figures are included to better illustrate the embodiments of the apparatus and technique of the present invention. The figures are not to scale and some features may be exaggerated for the purpose of illustrating certain aspects or embodiments of the present invention.

FIG. 1 is a perspective view of a microfluidic system including a microfluidic device in accordance with one embodiment of the invention.

FIG. 2 is a perspective view of a portion of a microfluidic channel of the microfluidic system of FIG. 1.

### DETAILED DESCRIPTION OF SPECIFIC EMBODIMENTS

Before describing the present invention in detail, it is to be understood that this invention is not limited to particular devices or biological systems, which can, of course, vary. It is also to be understood that the terminology used herein is for the purpose of describing particular embodiments only and is not intended to be limiting. As used in this specification and the appended claims, the singular forms “a”, “an” and “the” include plural referents unless the content clearly dictates otherwise. As used herein, the phrase “at least” means that the indicated item is equal to or greater than that designated value and the term “about” means that the designated value may vary by plus or minus ten percent, or nine percent, or eight percent, or seven percent, or six percent, or five percent, or four percent, or three percent, or two percent, or one percent. The term “substantially” varies with the context as understood by those skilled in the relevant art and generally means at least 70%, preferably means at least 80%, more preferably at least 90%, and most preferably at least 95%.

Embodiments of the present invention provide methods and devices for conducting point of care diagnostic testing as well as instrumental analyses. The methods and devices of some embodiments of the present invention comprise arrays of silicon CMOS sensors disposed in a channel that also comprises an array of features comprising binding partners for capturing analytes from a sample in a medium, which is flowed through the channel. The analytes are labeled with a luciferase that has a brightness that is at least one hundred times as great as firefly luciferase. With embodiments of the present invention, it is possible to carry out luminescent detection of analytes in a relatively simple device. The devices have a relatively small size and are lightweight and easily transportable, thus rendering the devices suitable for point-of-care applications. Because of relative small size, embodiments of the devices are particularly suited for conducting analyses on relatively small amounts of sample. Embodiments of the methods and devices have a sensitivity that is comparable to laboratory diagnostics. While particularly suited for point of care applications, some embodiments of the present invention are also applicable to diagnostic instrumentation.

As discussed above, some embodiments of the present invention are directed to methods for analyzing a sample for the presence of one or more analytes. The sample is contacted with a channel comprising (i) a plurality of features wherein each of the features comprises a binding partner for one of the respective analytes and (ii) a plurality of silicon CMOS sensors disposed such that each sensor senses a corresponding feature. The contacting is carried out under conditions for binding of an analyte to a respective binding partner. The analytes are treated to introduce a luciferase prior to or after the contacting. The luciferase has a brightness that is at least 100 times greater than firefly luciferase. Light emitted at each of the features is detected by means of the silicon CMOS sensors. The amount of light emitted at each of the features is related to the presence and/or amount of an analyte in the sample.

### Devices

Embodiments of devices in accordance with the present invention comprise a channel comprising a plurality of features, wherein each of the features comprises a binding partner for one of the respective analytes, and a plurality of silicon CMOS sensors disposed such that each sensor senses a corresponding feature.

The channel may be part of a microfluidic device or component of a microfluidic system. The term “microfluidic device” as used herein refers to a device having fluidic conduit features, such as, e.g., channels, that are difficult or impossible to see with the naked eye, that is, having features on a scale of millimeters to tenths of micrometers. The term microfluidic device refers to a device having a network of chambers connected by channels, in which the channels have mesoscale dimensions, e.g., having at least one cross-sectional dimension in the range from about 0.1 μm to about 500 μm. In microfluidic devices, micro-volumes of fluid are manipulated along a fluid flow path. “Micro-volume” means a volume from about 10 femtoliters to 500 μl, usually from about 100 femtoliters to about 200 μl.

The number of features in the microfluidic device is based on a number of factors such as, for example, the complexity of the sample to be analyzed including the suspected number of analytes, applications of the devices, e.g., portable vs. stationary, and so forth. The number of features may be more than ten, more than one hundred, more than five hundred, more than one thousand, more than fifteen hundred, more than two thousand, more than twenty five hundred features, more than 20,000, more than 25,000, more than 30,000, more than 35,000, more than 40,000, more than 50,000, more than 75,000, or more than 100,000. In many embodiments the number of features is in the range of about 100 to about 100,000 or more, about 1000 to about 100,000 or more, and so forth.

The microfluidic devices contain at least one fluid flow path through which fluid flows through the device, where a plurality of flow paths that may or may not be intersecting and may be positioned in any convenient configuration may be present in the device. Generally, the microfluidic devices have at least one chamber positioned at some point in the fluid flow path, where the term “chamber” means any type of structure in which micro-volumes of fluid may be contained, and includes micro-chambers, micro-channels, micro-conduits and the like. Depending on the nature of the chamber, the chamber may be the entire fluid flow path through the device, e.g., where the fluid flow path is a micro-channel, or may occupy only a portion of the fluid flow path of the device.

The term micro-chamber, as used herein, means any structure or compartment having a volume ranging from about 1 μl to 500 μl, having cross-sectional areas ranging from about 0.05 cm² with a chamber depth of 200 μm to 5 cm² with a chamber depth of 1 mm; or from about 10 μl to 500 μl, having a cross-sectional area ranging from about 0.5 cm² with a chamber depth of 200 μm to about 5 cm² with a chamber depth of 1 mm; or from about 20 μl to 200 μl, having a cross-sectional area ranging from about 1 cm² with a chamber depth of 200 μm to about 4 cm² with a chamber depth of 500 μm.

The chamber or channel structure may have any convenient configuration or cross-sectional shape, including square, circular, oval, trapezoidal, rectangular, octagonal, irregular, etc. Furthermore, the cross-section of the interior of a chamber or a channel may have several different cross-sectional shapes. For example, the cross-sectional shape of an area of the chamber or channel adjacent a pore or opening or orifice may be different than that of the remainder of the chamber.

Micro-channels or micro-conduits are chambers that are dimensioned or configured such that fluid is capable of flowing through the micro-channel by capillary flow, i.e., the micro-channel is of capillary dimensions. By capillary dimensions is meant a structure or container in which any cross-sectional dimension from one side to another, e.g., diameter, widest point between two walls of a channel, etc., does not exceed about 250 μm. Generally, for capillary flow, any cross-sectional dimension of the micro-channel will range from about 10 to 250 μm, usually from about 50 to 200 μm. The flow through the micro-channels may also be pressurized. Moving materials through microchannels may be accomplished by use of a fluid pressure difference and by use of various electro-kinetic processes including electrophoresis, electroosmotic flow, and electrokinetic pumping.

The micro-channel(s) of the device may have a linear configuration, a curved configuration, or any other configuration, e.g., spiral, angular, etc., or combinations thereof. In addition, as discussed above, there may be more than one micro-channel in the device, where the micro-channels may intersect at various points to form complicated flow paths or patterns through the device, e.g., Y-shaped intersections, T-shaped intersections, crosses; and/or be separated by one or more micro-chambers, etc.

In addition to a substrate that has features such as microfluidic channels, microfluidic compartments, and microfluidic flow control elements, the microfluidic component may include features such as capillary channels, separation channels, detection channels, valves and pumps. The microfluidic device may be a continuous or non-continuous flow device or a combination thereof. The devices also can include reservoirs, fluidly connected to the channels, which can be used to introduce material into the channels. Interfacing mechanisms, such as electropipettors, can be incorporated for transporting materials into wells or microfluidic channels.

In many embodiments, the micro-channel(s) of the microfluidic devices, as well as any other components, e.g., entry ports, etc., may be present in an essentially planar-shaped substrate, e.g., a card-shaped substrate, disk-shaped substrate, etc. The materials from which the chambers and related components may be fabricated are dependent on the particular environment or use of the chamber, the nature of the liquid within the chamber, the advantages and limitations of particular fabrication techniques, and so forth. Materials for fabrication include polymers, plastics such as polyimides, polycarbonates, polyesters, polyamides, polyethers, polyolefins, and mixtures thereof, resins, polysaccharides, silica or silica-based or silicon dioxide based materials such as quartz, fused silica, glass (borosilicates) etc., ceramics and composites thereof, carbon, metals including metal alloys, metal oxides, inorganic glasses, and so forth and combinations thereof. Particular plastics finding use include, for example, polyethylene, polypropylene, such as high density polypropylene, polytetrafluoroethylene (PTFE), e.g., TEFLON®, polymethylmethacrylate, polycarbonate, polyethylene terephthalate, polystyrene or styrene copolymers, polyurethanes, polyesters, polycarbonates, polyureas, polyamides, polyethyleneamines, polyarylene sulfides, polysiloxanes, polydimethylsiloxanes, polyimides, polyacetates, poly etheretherketone (PEEK), and the like. Metals include, for example, stainless steel, hastalloy, platinum, gold, silver, titanium, and so forth.

The microfluidic device or component may be fabricated by direct means such as photolithographic processes, wet or dry chemical etching, laser ablation, or traditional machining. The microfluidic component may also be fabricated by indirect means such as injection molding, hot embossing, casting, or other processes that utilize a mold or patterned tool to form the features of the microfluidic component.

The microfluidic devices may be fabricated as unitary devices or they may be constructed from several parts assembled into the device. Apertures may be made in the chamber housing by laser cutting, etching, piercing, drilling, punching, direct molding or casting from a master with pins, and so forth.

In one example, a microfluidic fluid delivery system may include a microfluidic device having a fluid input and a fluid reservoir. The aforementioned devices may also include means for introducing liquids into the devices as well as means for moving materials in the liquids within the devices and means for providing electrical control of functions of the microfluidic device.

In some embodiments the linear array is synthesized or deposited on an interior surface of a housing substrate and the area comprising at least the linear array is enclosed to form a channel comprising the linear array. Enclosure may be attained using an appropriate material to cover the channel and then sealing to form the housing. An apparatus may be fabricated using other convenient means, including conventional molding and casting techniques, extrusion sheet forming, calendaring, thermoforming, and the like.

Enclosing the housing to form the channel comprising the linear array may be accomplished in a number of ways. One important consideration in forming the linear array housing in general, and enclosing the housing in particular, is to avoid damage to the linear array on the surface of the housing substrate. In one approach, a separate material may be placed over the substrate comprising the linear array. The separate material is sealed to the substrate to enclose the housing to form the channel with the linear array therein. Sealing may be achieved by application of heat, adhesives, and so forth. The separate material may have the same composition as the substrate or a composition that is different from the substrate.

As mentioned above, the channel of the device comprises a plurality of features wherein each of the features comprises a binding partner for one of the respective analytes. The binding partners are bound to the interior surface of the channel in a non-diffusive manner. By the term “non-diffusive” is meant that the molecules that make up the individual features are bound to the surface in such a manner that they will not detach under the conditions of preparing and using the linear array. Non-diffusive binding may be covalent or may be non-covalent or macromolecular association where the linking is of sufficient strength to withstand the aforementioned conditions. Non-diffusive binding of the features may be achieved in a number of approaches known in the art. Some of those approaches are discussed briefly hereinbelow by way of illustration and not limitation.

The features generally are molecules that are involved in the detection of target molecules or analytes in a sample of interest. Each molecule of a feature may be specific for a corresponding analyte or for a compound indicative of the presence of the analyte. For example, the analyte may be part of a complex such as, for example, an antigen-antibody complex, polynucleotide-protein complex, polynucleotide-polynucleotide complex and the like, and the feature is capable of binding to a component of the complex. Usually, the molecule comprising the feature is a specific binding partner for the analyte or for a member of the complex indicative of the presence of the analyte. The members of a pair of molecules (e.g., a detector probe or a capture probe and a target analyte, or the members of a specific binding pair (e.g., antibody-antigen, nucleic acid, and protein-vitamin binding pairs)) are said to “specifically bind” to each other if they bind to each other with greater affinity than to other, non-specific molecules. For example, an antibody raised against an antigen to which it binds more efficiently than to a non-specific antigen can be described as specifically binding to the antigen. Similarly, a nucleic acid probe can be described as specifically binding to a nucleic acid target if it forms a specific duplex with the target by base pairing interactions.

The binding partner for the analyte depends on the nature of the analyte, which is discussed in more detail hereinbelow. Typical binding partners include, for example, antigens, antibodies, polynucleotide receptors, protein receptors, hormone receptors, enzymes, and the like.

Attaching the binding partner for the analyte to the interior surface of the channel to form the features may be accomplished in a number of different ways depending on the nature of the surface and the nature of the binding partner. The exposed surface of the channel either has a plurality of spots that comprise a functional group for attachment or must be treated or modified by chemical techniques to provide such spots with functional group or groups. Representative groups include, by way of illustration and not limitation, amino, especially primary amino, hydroxyl, thiol, sulfonic acid, phosphorous and phosphoric acid, particularly in the form of acid halides, especially chloride and bromide, and carboxyl, and the like.

A procedure for creating the attachment chemistry is sometimes referred to a “priming” the surface. To this end, the exposed surface is modified so as to prepare the surface for attachment of the binding partner. The binding partner may be attached directly to the exposed surface or it may be synthesized on the surface depending on the nature of the binding partner. In the former approach the binding partner comprises a functional group for attachment. In the latter approach the binding partner is formed in situ such as, for example, the formation of biopolymers by employing monomeric building blocks such as nucleotide triphosphates in the case of polynucleotides.

The exposed surface may be modified with groups or coupling agents to covalently link the binding partner. The reactive functional groups may be conveniently attached to the exposed surface through a hydrocarbyl radical such as an alkylene or phenylene divalent radical. Such hydrocarbyl groups may contain up to 10 carbon atoms, or up to 20 carbon atoms and the like.

In one embodiment, the surface of the interior of the channel is siliceous, i.e., the surface comprises silicon oxide groups, either present in the natural state, e.g., glass, silica, silicon with an oxide layer, etc., or introduced by techniques well known in the art. One technique for introducing siloxyl groups onto the surface involves reactive hydrophilic moieties on the surface. These moieties are typically epoxide groups, carboxyl groups, thiol groups, and/or substituted or unsubstituted amino groups as well as a functionality that may be used to introduce such a group such as, for example, an olefin that may be converted to a hydroxyl group by means well known in the art. One approach is disclosed in U.S. Pat. No. 5,474,796 (Brennan), the relevant portions of which are incorporated herein by reference. A siliceous surface may be used to form silyl linkages, i.e., linkages that involve silicon atoms. Usually, the silyl linkage involves a silicon-oxygen bond, a silicon-halogen bond, a silicon-nitrogen bond, or a silicon-carbon bond.

A procedure for the derivatization of a metal oxide surface uses an aminoalkyl silane derivative, e.g., trialkoxy 3-aminopropylsilane such as aminopropyltriethoxy silane (APS), 4-aminobutyltrimethoxysilane, 4-aminobutyltriethoxysilane, 2-aminoethyltriethoxysilane, and the like. APS reacts readily with the oxide and/or siloxyl groups on metal and silicon surfaces. APS provides primary amine groups that may be used to attach a binding partner to a feature location. Such a derivatization procedure is described in EP 0 173 356 B1, the relevant portions of which are incorporated herein by reference. Other methods for treating the surface will be suggested to those skilled in the art in view of the teaching herein.

In some embodiments a one-dimensional array of features is bound in a non-diffusive manner to a surface of the channel, which array may be referred to as a linear array. Each feature, or element, within the linear array is defined to be a small, regularly shaped region of the surface of the substrate. The features in the linear array are arranged in a predetermined manner. Each feature of a linear array usually carries a predetermined binding partner and is typically of homogeneous composition although in some circumstances mixtures may be employed. Each feature within the array may contain a different binding partner and some or all of the features may be of different compositions.

Each feature of the array may be separated by spaces or areas. Interarray areas and interfeature areas are usually present but are not essential. These interarray and interfeature areas do not carry any binding partner. Interarray areas and interfeature areas typically will be present where arrays are formed by the conventional in situ process or by deposition of previously obtained moieties, as described herein. It will be appreciated though that the interarray areas and interfeature areas, when present, could be of various sizes and configurations.

In the linear array the order of the features identifies each feature, which allows selective identification of target molecules or analytes. Usually, the linear array has a fixed length determined by the number of features of the linear array. The number of features is related to the nature of the features, the nature of the analytes, the complexity of the biological or clinical questions being investigated, the number of quality control features desired, and so forth. A typical linear array may contain more than about ten, more than about one hundred, more than about one thousand, more than about ten thousand, more than about twenty thousand, etc., more than about one hundred thousand, features and so forth. Usually, the number of features does not exceed about 10⁷ and or in some instances usually does not exceed about 10⁶. The density of the spots or features may also vary, where the density is generally at least about 1 spot/cm², or at least about 100 spots/cm², or in some embodiments at least about 400 spots/cm², where the density may be as high as 10⁶ spots/cm² or higher, or does not exceed about 10⁵ spots/cm², or does not exceed about 10⁴ spots/cm².

The width of the linear array is usually one feature. However, the width of the linear array may be greater than one feature where the size of the feature and the width of the housing, e.g., microchannel, permit. Therefore, the width of the linear array may be 1 to about 5 features, 1 to about 4 features, 1 to about 3 features, 1 to 2 features. In such an embodiment where the linear array is more than one feature wide, each feature comprising the width at the position in question may be the same or different and each feature comprising the length of the linear array may be the same or different, usually different, as discussed above. The width of the features, for example, the diameter of a round spot, may be in the range from about 10 μm to about 1.0 cm. In other embodiments each feature may have a width in the range of about 1.0 μm to about 1.0 mm, usually about 5.0 μm to about 500 μm, and more usually about 10 μm to about 200 μm. Non-round features may have width ranges equivalent to that of circular features with the foregoing width (diameter) ranges. The width of a feature may be larger, for example, where the feature comprises a higher concentration of binding partner than that of another feature.

The channel of embodiments of the present devices further comprises a plurality of silicon CMOS (complementary metal oxide semiconductor) sensors. CMOS sensors such as CMOS image sensors comprise an array of pixels each having a photodetector and devices for readout. Photons incident on the photodetector are converted into photocurrent. In a CMOS sensor, each pixel has its own charge-to-voltage conversion, and the sensor often also includes amplifiers, noise-correction, digitization circuits and the like.

The silicon CMOS sensors are disposed such that each sensor senses a corresponding feature. The sensors may be disposed in any fashion as long as a sensor can collect signal from a corresponding feature. In some embodiments the sensors are disposed on an interior surface of the channel that is opposite to the interior surface of the channel that comprises the features. The alignment of a particular sensor and a particular feature is such that the sensor can collect light emitted from the feature. In this way, the sensor and the feature are optically coupled. Accordingly, the sensor and the feature can be substantially axially aligned with one another or the axes may differ by an amount that is no greater than that which would result in insufficient detection of light from the feature to obtain the requisite sensitivity in an analysis. The amount that the two axes may differ is no more than about 10%, no more than about 5%, no more than about 4%, no more than about 3%, no more than about 2%, no more than about 1%, and so forth.

The number of silicon CMOS sensors usually corresponds to the number of features to be examined.

In some embodiments some or all of the silicon CMOS sensors comprise metallic nanoparticles. The metallic nanoparticles have a diameter of about 1 nanometer to about 500 nanometers micrometer. In alternative embodiments, nanoparticles of between about 1 nm to about 200 nm, about 5 nm to about 100 nm, about 10 nm to about 200 nm, about 20 nm to about 100 nm, about 30 nm to about 80 nm, about 40 nm to about 70 nm, or about 50 to about 60 nm, or the like in diameter are contemplated. In certain embodiments of the invention, nanoparticles with an average diameter of about 1 to about 100 nm, or about 10 to about 50 nm, about 50 to about 100 nm are contemplated.

In some embodiments of the invention, the metallic nanoparticles comprise a metal such as, for example, silver, gold, palladium, platinum, cobalt, nickel, chromium, copper, and mixtures and alloys thereof either with one or more of each other or with other suitable metals.

Nanoparticles are available commercially or they may be synthesized by procedures known in the art. Methods known for synthesizing metallic nanoparticles include mechanical methods, such as grinding large particles, and the like, and chemical methods such as, for example, reduction in which a reducing agent, such as sodium borohydride, is used to reduce a dissolved metal ion species to a metallic particle, and thin film methods such as evaporation of sputtering deposition, and so forth.

The shape of the nanoparticles may be approximately spherical, rod-like, edgy, faceted or pointy, although nanoparticles of any shape or of irregular shape may be used. In certain embodiments, the nanoparticles may be single nanoparticles and/or random aggregates of nanoparticles such as, e.g., colloidal nanoparticles or synthesized aggregates such as, e.g., dimers, trimers, tetramers or other aggregates.

The phrase “silicon CMOS sensors comprise (or comprising) metallic nanoparticles” means that the sensors and/or an area of the interior of the surface of the channel adjacent to the sensors have non-diffusively bound thereto metallic nanoparticles. Binding of the nanoparticles may be by adsorption, chemical bonding, and the like. In some embodiments, the nanoparticles may be modified to contain various reactive groups that may be employed to link the nanoparticles to the sensors and/or the interior surface of the channel. The type of linker compound used may be any compound that provides functionalization for linking. Linking groups that may be employed may be selected from those discussed above with regard to the attachment of a binding partner to the interior surface of the channel. In one approach the nanoparticles may be coated with derivatized silanes.

The density of the nanoparticles should be sufficient to enhance the sensitivity of the detection of photo-current from the silicon CMOS sensors. In some embodiments the density of the nanoparticles is about 0.1 particles/nm² to about 100 particles/nm², about 1 particles/nm² to about 10 particles/nm², about 10 particles/nm² to about 100 particles/nm², about 0.1 particles/nm² to about 10 particles/nm², about 1 particles/nm² to about 50 particles/nm², about 10 particles/nm² to about 50 particles/nm², and so forth.

The devices discussed above may include electronic and electrical processing support in the form of an electronic component to enhance the capabilities of the system. The devices may include, for example, electronic and electrical processing support that perform operations such as voltage/current sourcing, signal sourcing, signal detection, signal processing, signal feedback, and data processing separately from the microfluidic system. The electronic processing and microfluidic functions may be separated or may be integrated. For example, a relatively large power supply is required in order to apply a high voltage to a microfluidic channel for electrophoresis, and it is best to locate the power supply separate from the microfluidic system. As another example, data analysis is best performed using a computer that is separate from the microfluidic system.

In some embodiments, the electronics component may provide for individually electrically addressing and reading each of the silicon CMOS sensors. Accordingly, a device in accordance with the present invention may comprise a plurality of electrical leads coupled to each of the silicon CMOS sensors for electrically individually addressing the silicon CMOS sensors. The electrical leads may be formed by any technique and material typically used for electrical connections in a thin-film circuit, being patterned and/or multi-layered structures of metals, doped semiconductors, conductive organic films, and the like.

On-system electrical processing may be employed in cases where information gathered from many sensors on a microfluidic system must be used to control processes on the microfluidic chip. For example, a temperature system input might be used to control heaters of a microfluidic system.

In addition to microfluidic features, the microfluidic device or component may include conductive traces that are formed within the substrate and/or on the surface of the substrate. The conductive traces provide electrical connection between the electronics component and various electrical features on or in the microfluidic component. These electrical features may include: (1) direct contacts to fluid; (2) elements which, either in contact with or not in contact with fluid, control the flow or the operation of fluid or its contents; (3) sensors in direct contact with fluid; (4) sensors that do not directly contact fluid; (5) electrical heating or cooling elements integrated in or on the microfluidic component; (6) elements that can affect surface change within the microfluidic component; (7) active microfluidic control elements such as valves, pumps, and mixers; and so forth. Conductive traces may also lead to contact pads on the microfluidic component that provide electrical connections to off-component systems such as signal processors, signal readout devices, power supplies, and/or data storage systems. Providing contact pads on the microfluidic component for connection to off-component systems may eliminate the need to provide such contact pads on the electronics component.

While the electronics component may be composed of discrete electrical elements on a common substrate, such as a conventional printed circuit board, the component may be a prefabricated integrated circuit that may perform any of a variety of functions. The prefabricated integrated circuit may include a combination of op-amps, transistors, diodes, multiplexers, switches, filters, logic, digital-to-analog converters, analog-to-digital converters, etc., that perform functions such as signal detection, signal processing, buffering, and/or control functions such as, e.g., flow control and the like. The electronics component can be, for example, an application specific integrated circuit. As an alternative to the integrated circuit chip, the electronics component may consist of discrete electrical devices mounted on a suitable substrate, such as a printed circuit board, which may be integral or non-integral with the microfluidic component. The electronics component may be fabricated separately from the microfluidic component utilizing conventional semiconductor processing techniques.

The electronics component may include signal detection circuitry. The signal detection circuitry may detect signal in accordance with the present methods. It should be understood that circuitry for detecting other phenomena may also be included within the electronics component. The electronics component may also include signal processing circuitry. For example, the signal processing circuitry may amplify a signal, filter a signal, convert a signal from analog to digital, and make logical decisions based upon signal inputs. Because the possibilities for signal processing are numerous, it should be understood that any type of signal processing is anticipated for implementation in the electronics component consistent with the present methods involving detection of signal from the silicon CMOS sensors.

The electronics component may also provide circuitry for control functions such as voltage control, current control, temperature control, clock signal generation, etc. Flow control circuitry may be incorporated in order to manipulate microfluidic flow control elements of the type previously identified (e.g., valves, pumps, and regulators). As with the detection and processing circuitry, the possibilities for control circuitry are numerous and therefore it should be understood that any type of control circuitry is anticipated for implementation in the electronics component.

The electronics component may also contain software or firmware that, through its operation; guides or controls the action of the circuitry. For example, the electronics component may contain programmable logic that allows a programmed algorithm to be executed so as to perform certain functions. These functions may include signal filtration, signal feedback, control operations, signal interruption, and other forms of signal processing.

The electronics component may be fabricated in a separate operation utilizing either conventional semiconductor processing techniques or assembly of discrete electrical elements such as resistors, capacitors, operational amplifiers, and the like. The electronics component may include a combination of memory, signal detection, signal processing, and control circuitry. The control circuitry may provide voltage control, current control, temperature control, and/or clock signal generation. Where the electronics component is not integral with the microfluidic component, the electronics component can be bonded to the microfluidic component in various locations depending on the ease of manufacture and the like. In some embodiments, the electronic component may be maintained separate from the microfluidic device.

To assist in the automation of the present methods, the functions and methods may be carried out under computer control, that is, with the aid of a computer and computer program. The computer system is in communication with various components of the device and of the apparatus and the computer program product directs the components to carry out their respective functions.

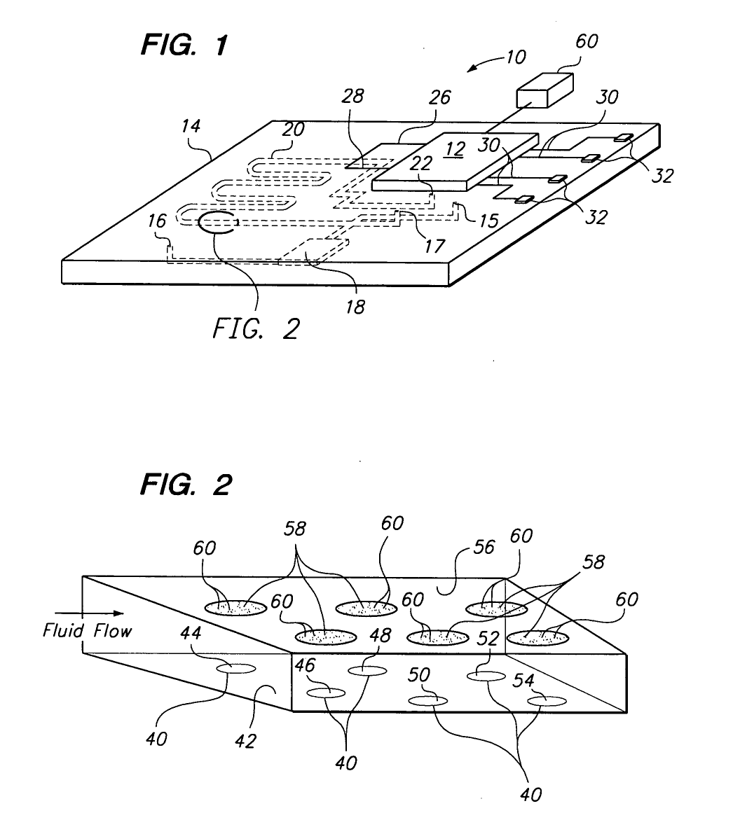

A specific embodiment of a device in accordance with embodiments of the present invention is shown in FIGS. 1 and 2. Referring specifically to FIG. 1, the microfluidic component 14 is a planar device that is part of apparatus 10 and includes chamber 18 having input/output ports 15 and 16 and further includes channel 20 having input/output ports 17 and 22. The chamber and channels are shown as dashed lines, since they are formed within the microfluidic component 14. The dashed lines are interrupted at the intersection of the channel from chamber 18 with channel 20 because the two channels intersect. Chamber 18 may be employed to carry out various sample preparation processes if required by a particular method. Such processes include, but are not limited to, mixing, labeling, filtering, extracting, precipitating, digesting, and the like. The microfluidic component also includes conductive traces 26, 28, and 30 that are formed within the substrate and/or on the surface of the substrate. For example, the conductive traces 26 and 28 may be used to assist in measure conductance as discussed above. The conductive traces 26 and 28 extend to the electronics component 12, which may be integral with or separate from microfluidic component 14. The microfluidic component also includes conductive traces 30 that connect the electronics component to contact pads 32. The contact pads may provide electrical connections to off-chip systems such as signal processors, signal readout devices, a power supply, and/or data storage systems as discussed above. Providing input/output contact pads on the microfluidic component is an alternative embodiment to providing such contact pads on the electronics component.

FIG. 2 shows a portion of channel 20, which comprises a plurality of features 40 disposed on an interior surface 42 of channel 20. Each of features 40 respectively comprise binding partners 44, 46, 48, 50, 52, 54 for analytes suspected of being present in a sample to be analyzed. Interior surface 56 of channel 20 comprises a plurality of silicon CMOS sensors 58 optically coupled to a respective feature 40. Each of sensors 58 is in electrical communication with electronics component 12. In the embodiment shown, each of sensors 58 comprises metallic nanoparticles 60.

The components of the present apparatus are adapted to perform a specified function usually by a combination of hardware and software. This includes the structure of the particular component and may also include a microprocessor, embedded real-time software and I/O interface electronics to control a sequence of operations and so forth.

The size of the overall device will depend on a number of factors such as the number of analytes, and thus the corresponding number of features, area required by electronics, the particular manner in which the device is used, and the like.

### Methods

As mentioned above, embodiments of the present invention are directed to methods for analyzing a sample for the presence of one or more analytes. The sample is contacted with a channel comprising (i) a plurality of features wherein each of the features comprises a binding partner for one of the respective analytes and (ii) a plurality of silicon CMOS sensors, each of the sensors being optically coupled to a corresponding feature. The contacting is carried out under conditions for binding of an analyte to a respective binding partner. The analytes are treated to introduce a luciferase prior to or after the contacting. The luciferase has a brightness that is at least 100 times greater than firefly luciferase. Light emitted at each of the features is detected by means of the silicon CMOS sensors. The amount of light emitted at each of the features is related to the presence and/or amount of an analyte in the sample.

The nature of the binding partners for the analytes is discussed above in detail. The analytes to be screened are the compounds or compositions to be detected. The analyte is usually a member of a specific binding pair (sbp) and the other member of the sbp is a binding partner for the analyte. The analyte or the binding partner may be a ligand, which is monovalent (monoepitopic) or polyvalent (polyepitopic), usually antigenic or haptenic, and is a single compound or plurality of compounds that share at least one common epitopic or determinant site. The analyte can be a part of a cell such as a bacterium or a cell bearing a blood group antigen such as A, B, D, etc., or an HLA antigen or the analyte may be a microorganism, e.g., bacterium, fungus, protozoan, or virus. In certain circumstances the analyte may also be a reference compound, a control compound, a calibrator, and the like.

The monoepitopic ligand analytes will generally be from about 100 to about 2,000 molecular weight, more usually, from about 125 to about 1,000 molecular weight. The monoepitopic analytes include drugs, e.g., drugs of abuse and therapeutic drugs, metabolites, pesticides, pollutants, nucleosides, and the like. Included among drugs of interest are the alkaloids, steroids, lactams, aminoalkylbenzenes, benzheterocyclics, purines, drugs derived from marijuana, hormones, vitamins, prostaglandins, tricyclic antidepressants, anti-neoplastics, aminoglycosides, antibiotics, nucleosides and nucleotides, miscellaneous individual drugs which include methadone, meprobamate, serotonin, meperidine, lidocaine, procainamide, acetylprocainamide, propranolol, griseofulvin, valproic acid, butyrophenones, antihistamines, chloramphenicol, anticholinergic drugs, such as atropine, their metabolites and derivatives, and so forth.

Metabolites related to diseased states include spermine, galactose, phenylpyruvic acid, and porphyrin Type 1 and so forth.

Among pesticides of interest are polyhalogenated biphenyls, phosphate esters, thiophosphates, carbamates, polyhalogenated sulfenamides, their metabolites and derivatives.

The polyvalent ligand analytes will normally be poly(amino acids), i.e., polypeptides and proteins, polysaccharides, mucopolysaccharides, nucleic acids, and combinations thereof. Such combinations include components of bacteria, viruses, chromosomes, genes, mitochondria, nuclei, cell membranes and the like.

A polynucleotide or nucleic acid is a compound or composition that is a polymeric nucleotide or nucleic acid polymer, which may include modified nucleotides.

For the most part, the polyepitopic ligand analytes to which the subject invention can be applied have a molecular weight of at least about 5,000, more usually at least about 10,000. In the poly(amino acid) category, the poly(amino acids) of interest will generally be from about 5,000 to 5,000,000 molecular weight, more usually from about 20,000 to 1,000,000 molecular weight; among the hormones of interest, the molecular weights will usually range from about 5,000 to 60,000 molecular weight.

A wide variety of proteins may be considered as to the familyof proteins having similar structural features, proteins having particular biological functions, proteins related to specific microorganisms, particularly disease causing microorganisms, etc. Such proteins include, for example, immunoglobulins, cytokines, enzymes, hormones, cancer antigens, nutritional markers, tissue specific antigens, etc. Such proteins include, by way of illustration and not limitation, protamines, histones, albumins, globulins, scleroproteins, phosphoproteins, mucoproteins, chromoproteins, lipoproteins, nucleoproteins, glycoproteins, T-cell receptors, proteoglycans, HLA, unclassified proteins, e.g., somatotropin, prolactin, insulin, pepsin, proteins found in human plasma, blood clotting factors, protein hormones such as, e.g., follicle-stimulating hormone, luteinizing hormone, luteotropin, prolactin, chorionic gonadotropin, tissue hormones, cytokines, cancer antigens such as, e.g., PSA, CEA, a-fetoprotein, acid phosphatase, CA19.9 and CA125, tissue specific antigens, such as, e.g., alkaline phosphatase, myoglobin, CPK-MB and calcitonin, and peptide hormones. Other polymeric materials of interest are mucopolysaccharides and polysaccharides.

For receptor analytes, the molecular weights will generally range from 10,000 to 2×10⁸, more usually from 10,000 to 10⁶. For immunoglobulins, IgA, IgG, IgE and IgM, the molecular weights will generally vary from about 160,000 to about 10⁶. Enzymes will normally range from about 10,000 to 1,000,000 in molecular weight. Natural receptors vary widely, generally being at least about 25,000 molecular weight and may be 10⁶ or higher molecular weight, including such materials as avidin, DNA, RNA, thyroxine binding globulin, thyroxine binding prealbumin, transcortin, etc.

The term analyte further includes polynucleotide analytes such as those polynucleotides defined below. These include m-RNA, r-RNA, t-RNA, DNA, DNA-RNA duplexes, etc. The term analyte also includes receptors that are polynucleotide binding agents, such as, for example, restriction enzymes, activators, repressors, nucleases, polymerases, histones, repair enzymes, chemotherapeutic agents, and the like.

Also included within the term “analyte” are polysaccharides or carbohydrates, lipids, fatty acids and the like.

The analyte may be a biomarker, which is a biochemical feature or facet that can be used to measure the progress of a disease or illness or the effects of treatment of a disease or illness. The biomarker may be, for example, a virus, a bacteria, a cancer antigen, a heart disease indicator, a stroke indicator, an Alzheimer's disease indicator, and the like.

The analyte may be a molecule found directly in a sample such as biological tissue, including body fluids, from a host. The sample can be examined directly or may be pretreated to render the analyte more readily detectable. Furthermore, the analyte of interest may be determined by detecting an agent probative of the analyte of interest such as a specific binding pair member complementary to the analyte of interest, whose presence will be detected only when the analyte of interest is present in a sample. Thus, the agent probative of the analyte becomes the analyte that is detected in an assay. The biological tissue includes excised tissue from an organ or other body part of a host and body fluids.

The sample may be a “body fluid sample” or a “non-body fluid sample.” The phrase “body fluid sample” refers to any fluid obtained from the body of a mammal (e.g., human, monkey, mouse, rat, rabbit, dog, cat, sheep, cow, pig, and the like), bird, reptile, amphibian or fish that is suspected of containing a particular target analyte or analytes to be detected. Exemplary body fluid samples for detection herein can be selected from one or more of whole-blood, plasma, serum, interstitial fluid, sweat, saliva, urine, semen, stool, sputum, cerebral spinal fluid, tears, mucus, blister fluid, inflammatory exudates, and the like. Also explicitly contemplated herein as a “body fluid sample” are body gas and body vapor. The phrase “non-body fluid sample” refers to any fluid not obtained from the body of a mammal, bird, reptile, amphibian or fish, which is suspected of containing a particular target analyte or analytes to be detected. Exemplary non-body fluid samples include cell culture media, artificial collection fluid, dialysate, and the like. An artificial collection fluid (or extraction fluid) can be prepared by bathing a particular surface area of an animal or an inanimate object with a fluid to collect into the fluid an endogenous or exogenous analyte for detection.

For determining a mixture of analytes such as, for example, proteins, one may use intact cells, intact viruses, viral infected cells, lysates, plasmids, mitochondria or other organelles, fractionated samples, or other aggregation of analytes, separated analytes, and treated analytes, by themselves or in conjunction with other compounds. Any source of a mixture of analytes can be used, where there is an interest in identifying a plurality of analytes. Analytes may be released and/or isolated using precipitation, extraction, lysing, chromatographic separation, and so forth and combinations thereof. The analytes may be present as individual analytes or combined in various aggregations, such as organelles, cells, viruses, etc.

Each of the analytes comprises a bioluminescent label. Accordingly, the analytes are treated to introduce a bioluminescent label prior to or after contacting a medium suspected of containing the analytes with the channel comprising the silicon CMOS sensors and the features having binding partners for the analytes attached thereto.

Bioluminescence is a luminescence phenomenon in which energy is specifically channeled to a molecule to produce an excited state and involves the use of molecular oxygen, either bound or free in the presence of a luciferase. Luciferases are oxygenases that act on a substrate, luciferin, in the presence of molecular oxygen and transform the substrate to an excited state. Upon return to a lower energy level, energy is released in the form of light. Luciferase refers to an enzyme or photoprotein that catalyzes a reaction that produces bioluminescence. The luciferase is a protein that occurs naturally in an organism or a variant or mutant thereof, such as a variant produced by mutagenesis that has one or more properties, such as thermal or pH stability, that differ from the naturally-occurring protein.

The luciferase employed in the present methods has a brightness that is greater than that of firefly luciferase as measured under the same conditions. The brightness is at least 100 times greater, or at least 200 times greater, or at least 300 times greater, or at least 400 times greater, or at least 500 times greater, and so forth, than firefly luciferase. By the term “brightness” is meant the amount of light emitted by the luciferase under the conditions of the analyses in accordance with the present methods. Brightness is determined using, for example, a conventional luminometer.

In some embodiments the luciferase is Gaussia marine luciferase. Gaussia marine luciferase is commercially available or is isolatable or synthesizable. The Gaussia marine luciferase may be isolated from the corresponding marine organism by techniques that are known in the art. On the other hand, nucleic acids may be isolated from the organism and used to prepare Gaussia marine luciferase.

As mentioned above, in some embodiments the analytes are treated to attach a respective bioluminescent label prior to or after being exposed to the channel comprising binding partners for the respective analytes that might be present in a medium to be analyzed. Attachment of a bioluminescent label to an analyte may be accomplished directly or indirectly, covalently or non-covalently. Covalent attachment may be by a bond (direct attachment) or a linking group (indirect attachment). In either case, covalent attachment normally involves one or more functional groups on the bioluminescent label and/or the analyte. In embodiments where a linking group is involved, the linking group varies depending upon the nature of the molecules, i.e., the bioluminescent label or the analyte. Functional groups that are normally present or are introduced on the molecules to be attached are employed for linking these materials.

Alternative functionalities of oxo include active halogen, diazo, mercapto, olefin, particularly activated olefin, amino, phosphoro and the like. The linking groups may vary from a bond to a chain of from 1 to 100 atoms, usually from about 1 to 70 atoms, preferably 1 to 50 atoms more preferably 1 to 20 atoms, each independently selected from the group normally consisting of carbon, oxygen, sulfur, nitrogen, halogen and phosphorous. The number of heteroatoms in the linking groups will normally range from about 0 to 20, usually from about 1 to 15, more preferably 2 to 6. The atoms in the chain may be substituted with atoms other than hydrogen in a manner similar to that described above for the substituent having from 1 to 50 atoms. As a general rule, the length of a particular linking group can be selected arbitrarily to provide for convenience of synthesis and the incorporation of the desired bioluminescent label. The linking groups may be aliphatic or aromatic, although with diazo groups, aromatic groups will usually be involved.

When heteroatoms are present, oxygen will normally be present as oxo or oxy, bonded to carbon, sulfur, nitrogen or phosphorous, nitrogen will normally be present as nitro, nitroso or amino, normally bonded to carbon, oxygen, sulfur or phosphorous; sulfur would be analogous to oxygen; while phosphorous will be bonded to carbon, sulfur, oxygen or nitrogen, usually as phosphonate and phosphate mono- or diester.

Common functionalities in forming a covalent bond between the linking group and the molecule to be conjugated are alkylamine, amidine, thioamide, ether, urea, thiourea, guanidine, azo, thioether and carboxylate, sulfonate, and phosphate esters, amides and thioesters. For the most part, carbonyl functionalities will find use, both oxocarbonyl, e.g., aldehyde, and non-oxocarbonyl (including nitrogen and sulfur analogs) e.g., carboxy, amidine, amidate, thiocarboxy and thionocarboxy.

In some embodiments, the linking group has a non-oxocarbonyl group including nitrogen and sulfur analogs, a phosphate group, an amino group, alkylating agent such as halo or tosylalkyl, oxy (hydroxyl or the sulfur analog, mercapto) oxocarbonyl (e.g., aldehyde or ketone), or active olefin such as a vinyl sulfone or α-, β-unsaturated ester. These functionalities will be linked to amine groups, carboxyl groups, active olefins, alkylating agents, e.g., bromoacetyl. Where an amine and carboxylic acid or its nitrogen derivative or phosphoric acid are linked, amides, amidines and phosphoramides will be formed. Where mercaptan and activated olefin are linked, thioethers will be formed. Where a mercaptan and an alkylating agent are linked, thioethers will be formed. Where aldehyde and an amine are linked under reducing conditions, an alkylamine will be formed. Where a carboxylic acid or phosphate acid and an alcohol are linked, esters will be formed.

Non-covalent attachment of a bioluminescent label may involve a bioluminescent label being bound to a binding partner for the analyte such as, for example, an antibody or other receptor for the analyte, and the like. The binding partner chosen for attachment of a bioluminescent label is normally different from the binding partner that is attached to the channel. The two binding partners at least should be different enough to bind to different sites on the analyte.

The binding partner with the bioluminescent label attached may be combined with the analyte prior to or after contact of the medium suspected of containing the analyte with the channel comprising binding partners for the analytes at the respective feature sites. As a further alternative, the analyte, for example, may be bound by a first antibody specific to the analyte, while the bioluminescent label is a labeled second antibody specific to the first antibody. Other approaches will be suggested to one skilled in the art in light of the present disclosure.

The concentration of analytes to be detected will generally vary from about 10⁻⁵ to 10⁻¹⁷ M, more usually from about 10⁻⁶ to 10⁻¹⁴ M.

The medium suspected of containing the analytes, which may or may not comprise a bioluminescent label, is contacted with the channel comprising the features and the silicon CMOS sensors. In some embodiments the medium is an aqueous medium and in other embodiments the medium is a non-aqueous medium. The nature of the medium depends on the nature of the analytes and the like.

An aqueous medium may be solely water or may include from 0.01 to 80 or more volume percent of a cosolvent such as an organic solvent, which may be polar or non-polar, usually polar for purposes of solubility. Examples of polar organic solvents include oxygenated organic solvents of from 1 to about 30 carbon atoms, or 1 to about 20 carbon atoms, or 1 to about 10 carbon atoms including alcohols, ethers, ketones, aldehydes, amides, nitrites, and so forth. Particular examples include alcohols such as, e.g., ethoxyethanol, ethanol, ethylene glycol and benzyl alcohol; amides such as dimethyl formamide, formamide, acetamide and tetramethyl urea and the like; sulfoxides such as dimethyl sulfoxide and sulfolane; nitriles such as, e.g., acetonitrile, and so forth, ethers such as carbitol, ethyl carbitol, dimethoxyethane, and the like. Non-polar solvents include, for example, hydrocarbons containing 1 to about 30 carbon atoms, or 1 to about 20 carbon atoms, or 1 to about 10 carbon atoms, and so forth; halogenated hydrocarbons such as, e.g., methylene chloride, trichloromethane carbon tetrachloride, and so forth.

When an aqueous medium is employed, it is generally an aqueous buffered medium that is buffered at a moderate pH, generally that which provides optimum sensitivity and specificity for a particular analyses. The pH for the medium will usually be in the range of about 4 to 13, more usually in the range of about 5 to 10, and preferably in the range of about 6.5 to 9.5. Various buffers may be used to achieve the desired pH and maintain the pH during the determination. Illustrative buffers include borate, phosphate, carbonate, tris, barbital and the like. The particular buffer employed is not critical, but in an individual analyses one or another buffer may be preferred.

As mentioned above, the medium suspected of containing the analytes, which may or may not comprise a bioluminescent label, is contacted with the channel of the present device. In some embodiments the channel is part of a microfluidic device and takes the form of one or more chambers or channels within the microfluidic device. In many embodiments, the medium is introduced into the microfluidic device by means of capillary action. However, as mentioned above, other forms of introduction may be employed such as, for example, positive or negative pressure, and the like

The contacting is carried out under conditions for binding of an analyte to a respective binding partner. Moderate temperatures are normally employed and, in many embodiments, the temperature is usually a constant temperature. The temperatures for binding will normally range from about 5° to about 99° C., about 15° to about 70° C., about 20 to about 45° C. Temperatures during measurements will generally range from about 10° to about 70° C., about 20° to about 45° C., about 20° to about 25° C. It will be appreciated, however, that higher or lower temperatures may be employed depending on the nature of the analytes, binding partners, medium, and the like.

The medium may also contain or be followed by reagents required for producing the bioluminescence reaction. Thus, in addition to the particular luciferase, luciferin and other substrates, solvents and other reagents that may be required to complete a bioluminescent reaction. Appropriate reaction conditions may be necessary for a bioluminescence reaction to occur. Such conditions include, for example, pH, salt concentrations and temperature. The particular nature of the reagents and conditions suitable for producing bioluminescence depend on the nature of the luciferase, and the like. Activators necessary to complete the bioluminescence reaction, such as oxygen and a substrate with which the luciferase reacts in the presence of the oxygen to produce light may also be included.

Following exposure of the medium to the channel and incubation under conditions for binding of the analytes to respective binding partners attached to the features of the channel, a wash fluid may be introduced into and flowed through the microfluidic device to remove unbound materials. However, in some instances a wash fluid is not required because each feature in combination with a respective silicon CMOS sensor provides for its own localized detection site.

Subsequent to the binding reactions and activation of the bioluminescent labels, light emitted at each of the features is detected by means of the silicon CMOS sensors. In many embodiments the amount of light emitted at each of the features is related to the presence and/or amount of an analyte in the sample.

In that regard, an electronic component as discussed above may be employed to assist in the detection of the light based on the binding of an analyte to a respective binding partner and/or communication of signals that are detected to a centralized data collector. Signals may be conveyed to a central computer where the signals are analyzed and related to the presence of an analyte at a particular feature. An increase in light intensity at a feature generally correlates with the presence of an analyte bound to a respective binding partner. In many embodiments the identity of each binding partner at each feature is known so that the analytes may be differentially detected and/or quantitated. Quantitation may be realized by measuring the amount of light from each feature and relating the amount of light, or the amount of variation in light emitted, to the presence and/or amount of analyte in the sample.

A particular embodiment of a method in accordance with the present invention will be discussed, by way of illustration and not limitation, with reference to FIGS. 1 and 2. A sample suspected of containing one or more analytes is treated to introduce a Gaussia marine luciferase label on each of the analytes. A medium comprising the sample treated as above is contacted with port 16 of microfluidic system 10 and allowed to flow into microfluidic device 14. The medium travels along a flow path 55 defined by channel 20 and analytes, if present, from the medium bind to respective binding partners, a portion of which includes binding partners 44, 46, 48, 50, 52, 54 attached at respective features 40. For purposes of this example, assume that an analyte is present that binds to binding partners 46, 48, 50, 52 so that a Gaussia luciferase labeled analyte is bound at features 40 that comprise binding partners 46, 48, 50, 52, respectively. The flow rate, temperature and the like of the medium are sufficient to permit the binding reactions to occur.

Following the binding of the analytes to respective binding partners and the passage of a wash fluid if necessary and introduction of a medium comprising reagents for carrying out a bioluminescent reaction and appropriate incubation conditions, sensors 58 are employed to detect light from respective features 40. Each of the excited Gaussia luciferase labels emits light that is detected by a respective sensor 58, which is in electronic communication with electronic component 12. The signal is relayed to computer 60, which correlates the signal to the presence and/or amount of the respective analytes in the sample and provides an appropriate read-out of data.

### Apparatus

As mentioned above, one embodiment of the present invention is an apparatus comprising a microfluidic system including a microfluidic device as described above, a computer system, which comprises a computer, for controlling the mechanism for correlating light detected by the silicon CMOS sensors to the presence and/or amount of an analyte in the sample, and (c) a computer program on a computer readable medium for controlling the computer.

The computer may be, for example, an IBM® compatible personal computer (PC) and the like. The computer is driven by software specific to the methods described herein. Software that may be used to carry out the methods may be, for example, Microsoft Excel or Microsoft Access and the like, suitably extended via user-written functions and templates, and linked when necessary to stand-alone programs that perform other functions. The computer system is in communication with various components of the device and of the apparatus and the computer program product directs the components to carry out their respective functions.

The computer system may be programmed from a computer readable storage medium that carries code for the system to execute the steps required of it, thus, having programming stored thereon for implementing the subject methods. The computer readable media may be, for example, in the form of a computer disk or CD, a floppy disc, a magnetic “hard card”, a server, or any other computer readable media capable of containing data or the like, stored electronically, magnetically or optically and including, for example, machine readable bar code, solid state electronic storage devices such as random access memory (RAM), or read only memory (ROM), or any other physical device or medium that might be employed to store a computer program. It will also be understood that computer systems of the present invention can include the foregoing programmable systems and/or hardware or hardware/software combinations that can execute the same or equivalent steps. Accordingly, stored programming embodying steps for carrying-out the subject methods may be transferred to a computer such as a personal computer (PC), (i.e., accessible by a researcher or the like), by physical transfer of a CD, floppy disk, or like medium, or may be transferred using a computer network, server, or other interface connection, e.g., the Internet.

The computer program product, therefore, comprises a computer readable storage medium having a computer program stored thereon which, when loaded into a computer, performs the aforementioned method and/or controls the functions of the aforementioned apparatus.

The computer program is designed to carry out a method for analyzing a sample for the presence of one or more analytes. The computer program provides for carrying out steps in a method wherein a sample is contacted with a channel, such as by flowing therethrough, wherein the channel comprises a plurality of features, each of the features comprises a binding partner for one of the respective analytes. The contacting is carried out under conditions for binding of an analyte to a respective binding partner and wherein a plurality of silicon CMOS sensors disposed within the channel is employed to sense light from respective features. The light collected at each of the features is then employed to determine the presence and/or amount of one or more analytes in the sample.

An embodiment of an apparatus in accordance with the present invention is depicted in FIG. 1 by way of illustration and not limitation. Apparatus 10 comprises microfluidic component 14, electronics component 12 and computer 60.

All publications and patent applications cited in this specification are herein incorporated by reference as if each individual publication or patent application were specifically and individually indicated to be incorporated by reference, except insofar as they may conflict with those of the present application (in which case the present application prevails). Methods recited herein may be carried out in any order of the recited events, which is logically possible, as well as the recited order of events.

The aforementioned description includes theories and mechanisms by which the invention is thought to work. It should be noted, however, that such proposed theories and mechanisms are not required and the scope of the present invention should not be limited by any particular theory and/or mechanism.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims. Furthermore, the foregoing description, for purposes of explanation, used specific nomenclature to provide a thorough understanding of the invention. However, it will be apparent to one skilled in the art that the specific details are not required in order to practice the invention. Thus, the foregoing descriptions of specific embodiments of the present invention are presented for purposes of illustration and description; they are not intended to be exhaustive or to limit the invention to the precise forms disclosed. Many modifications and variations are possible in view of the above teachings. The embodiments were chosen and described in order to explain the principles of the invention and its practical applications and to thereby enable others skilled in the art to utilize the invention.

1. A method for analyzing a sample for the presence of one or more analytes, said method comprising:
(a) contacting the sample with a channel comprising (i) a plurality of features wherein each of the features comprises a binding partner for one of the respective analytes and (ii) a plurality of silicon CMOS sensors. each of the sensors being optically coupled to a corresponding feature, and wherein the contacting is carried out under conditions for binding of an analyte to a respective binding partner and wherein the analytes are treated to introduce a luciferase prior to or after the contacting and wherein the Iuciferase has a brightness that is at least 100 times greater than firefly luciferase, and
(b) detecting light emitted from each of the features by means of the silicon CMOS sensors wherein the light emitted at each of the features is related to the presence and/or amount of an analyte in the sample.

2. A method according to claim 1 wherein the luciferase is a bioluminescent marine luciferase.

3. A method according to claim 1 wherein the luciferase is a Gaussia Juciferase.

4. A method according to claim 1 wherein the silicon CMOS sensors comprise metallic nanoparticles.

5. A method according to claim 1 wherein the channel is present in a substrate comprising silicon, glass or polymer or mixtures thereof.

6. A method according to claim 1 wherein the channel is part of a microfluidic system.

7. A method according to claim 6 wherein the contacting is carried out by flowing the sample through the channel.

8. A method according to claim 1 wherein the analytes are selected from the group consisting of small organic compounds, proteins, peptides, higher molecular weight carbohydrates, polynucleotides, fatty acids and lipids.

9. A method for analyzing a sample for the presence of one or more analytes. said method comprising:
(a) contacting the sample with a channel of a microfluidic system wherein the channel comprises (i) a plurality of features wherein each of the features comprises a binding partner for one of the respective analytes and (ii) a plurality of silicon CMOS sensors, each of the sensors being optically coupled to a corresponding feature. Wherein the contacting is carried out under conditions for binding of an analyte to a respective binding partner and wherein the analytes are treated to introduce a Gaussia Iuciferase prior to or after the contacting, and
(b) detecting light emitted from each of the features by means of the silicon CMOS sensors wherein the light emitted at each of the features is related to the presence and/or amount of an analyte in the sample.

10. A method according to claim 9 wherein the silicon CMOS sensors comprise metallic nanoparticles.

11. A method according to claim 9 wherein the microfluidic system comprises a substrate comprising silicon, glass, polymer, and mixtures thereof.

12. A method according re claim 9 wherein the analytes are selected from the group consisting of small organic compounds, polypeptides, peptides, higher molecular weight carbohydrates, polynucleotides, fatty acids and lipids.

13. A method according to claim 9 wherein the analytes are biomarkers.

14. A method according to claim 13 wherein the biomarkers are selected from the group consisting of viruses, bacteria and cancer antigens.

15. A device for analyzing a sample for the presence of one or more analytes, said device comprising:
(a) a channel comprising (i) a plurality of features wherein each of the features comprises a binding partner for one of the respective analytes and wherein one or more analytes are bound to a respective binding partner wherein each analyte comprises a bioluminescent marine luciferase that has a brightness that is at least 100 times greater than firefly luciferase, and (ii) a plurality of silicon CMOS sensors, each of the sensors being optically coupled to a corresponding feature, and
(b) a mechanism for correlating light detected by the silicon CMOS sensors to the presence and/or amount of an analyte in the sample.

16. A device according to claim 15 wherein the bioluminescent marine luciferase is a Gaussia luciferase.

17. A device according to claim 15 wherein the channel is present in a substrate comprising silicon, glass or polymer or mixtures thereof

18. A device according to claim 15 wherein the channel is part of a microfluidic system.

19. A device according to claim 15 wherein the silicon CMOS sensors comprise metallic nanoparticles.

20. An apparatus comprising:
(a) a device according to claim 15,
(b) a computer system for controlling the mechanism for correlating light detected by the silicon CMOS sensors to the presence and/or amount of an analyte in the sample, and
(c) a computer program on a computer readable medium for controlling the computer system.

21. The method according to claim 1, wherein said sensors are disposed on an interior surface of said channel that is opposite to an interior surface of said channel comprising said features.

22. The method according to claim 1, wherein each of said sensors is substantially axially aligned with said corresponding feature such that their axes differ by no more than about 10%.

23. The method according to claim 1, wherein a wash step is not performed following said contacting step or prior to said detecting step.
